(19) United States
(12) Patent Application Publication
(10) Pub. No.: US 2012/0059225 A1
(43) Pub. Date: Mar. 8, 2012

(54) EXPANDABLE SPACE-OCCUPYING TISSUE RETRACTORS

(75) Inventors: Christopher J. Gostout, Rochester, MN (US); Juliane Bingener-Casey, Rochester, MN (US); Eduardo Aimore Bonin, Rochester, MN (US)

(73) Assignee: Mayo Foundation for Medical Education and Research, Rochester, MN (US)

(21) Appl. No.: 13/224,100

(22) Filed: Sep. 1, 2011

Related U.S. Application Data
(60) Provisional application No. 61/380,124, filed on Sep. 3, 2010.

(51) Int. Cl. A61B 1/32

(52) U.S. Cl. 600/204

(57) ABSTRACT

This document provides methods and materials for retracting tissue during a minimally invasive surgical procedure (e.g., an endoscopic surgical procedure). For example, methods and materials for using an expandable space-occupying tissue retractor during an endoscopic surgical procedure such as a laparoscopic cholecystectomy procedure or a NOTES procedure are provided.

### CROSS-REFERENCE TO RELATED APPLICATIONS

This application claims the benefit of U.S. Provisional Application Ser. No. 61/380,124, filed Sep. 3, 2010. The disclosure of the prior application is considered part of (and is incorporated by reference in) the disclosure of this application.

### BACKGROUND

1. Technical Field

This document relates to methods and materials involved in retracting tissue during a minimally invasive surgery (e.g., a robotic, laparoscopic, or endoscopic surgical procedure). For example, this document relates to methods and materials for using an expandable space-occupying retractor during a robotic, laparoscopic, or endoscopic surgical procedure such as a laparoscopic cholecystectomy procedure or a natural orifice transluminal endoscopic surgery (NOTES) procedure.

2. Background Information

Endoscopic surgeries involve using scopes (e.g., endoscopes, laparoscopes, and robotic imaging devices) to go through small incisions or natural body openings in order to diagnose and/or treat medical conditions and diseases. Endoscopic surgeries can be described as being a minimally invasive surgery, which emphasizes that diagnosis and treatment can be done with reduced body cavity invasion. NOTES is a type of endoscopic surgery that involves performing a “scarless” operation (e.g., an abdominal operation) by passing an endoscope through a natural orifice (e.g., mouth, vagina, anus, etc.) then through an internal incision in, for example, the esophagus, stomach, vagina, bladder, or colon. This procedure avoids any external incisions or scars.

### SUMMARY

This document provides methods and materials for retracting tissue during a minimally invasive surgical procedure (e.g., an endoscopic surgical procedure). For example, this document provides methods and materials for using an expandable space-occupying tissue retractor during an endoscopic surgical procedure such as a laparoscopic cholecystectomy procedure, a NOTES procedure, or a robotic procedure. As described herein, a tissue retractor provided herein can be deployed and retrieved without adding an extra access point (e.g., without adding an extra parietal access), can allow adequate force to be applied to tissue for multidirectional organ displacement with minimal contact pressure, and can ensure optimal tissue exposure for the performance of surgical tasks such as tissue dissecting, hemostasis, and suturing. In some cases, a tissue retractor provided herein can, upon deployment, expand to form a bulb-shaped cap structure having a distal opening. The tissue retractor can be attached to the tip region of a rigid or flexible endoscopic device and can be configured to allow for navigation and tissue retraction of structures around the field of view.

In general, one aspect of this document features a tissue refractor configured for deployment from a tubular component during a minimally invasive surgical procedure (e.g., a minimally invasive endoscopic surgical procedure). The tissue retractor comprises, or consists essentially of, an expandable portion, wherein the expandable portion is in a compressed configuration when located within the tubular component, wherein the expandable portion is in an expanded configuration when advanced out of the tubular component, wherein the expanded configuration is capable of retracting tissue at a surgical site during the endoscopic procedure, wherein the expandable portion defines an opening at a distal end region of the expandable portion, wherein at least a portion of the expandable portion comprises transparent material configured to allow visualization of at least a portion of the tissue being retracted by the expandable portion, wherein an endoscopic tool is capable of advancing through the expanded region of the expandable portion when the expandable portion is in the expanded configuration and through the opening into contact with tissue at the surgical site. The tissue retractor can comprise struts. The tissue retractor can comprise a ring structure. The expandable portion of the tissue retractor can have a maximum diameter of between about 2.5 cm and about 5.0 cm when in the expanded configuration. The expandable portion of the tissue retractor can have a maximum diameter of between about 3.0 cm and about 4.0 cm when in the expanded configuration. The expandable portion of the tissue retractor can have a length of between about 2.0 cm and about 10.0 cm when in the expanded configuration. The expandable portion of the tissue retractor can have a length of between about 2.5 cm and about 5.0 cm when in the expanded configuration. The opening can have a maximum diameter of between about 1.0 cm and about 2.0 cm when in the expandable portion is in the expanded configuration.

In another aspect, this document provides a tissue retractor configured for deployment from a tubular component during a minimally invasive surgical procedure. The tissue retractor comprises, or consists essentially of, a plurality of wire structures configured to form at least part of an expandable portion and at least part of a tubular portion, wherein the expandable portion is in a compressed configuration when located within the tubular component, wherein the expandable portion is in an expanded configuration when advanced out of the tubular component, wherein the expanded configuration is capable of retracting tissue at a surgical site during the minimally invasive surgical procedure, wherein the expandable portion defines an opening at a distal end region of the expandable portion, wherein an endoscopic tool is capable of advancing through the expanded region of the expandable portion when the expandable portion is in the expanded configuration and through the opening into contact with tissue at the surgical site. The tissue refractor can comprise a positioning structure configured to maintain the positioning of the plurality of wire structures. The positioning structure and at least a portion of the plurality of wire structures can form the entirety of the expandable portion. The expandable portion of the tissue retractor can have a maximum diameter of between about 2.5 cm and about 5.0 cm when in the expanded configuration. The expandable portion of the tissue retractor can have a maximum diameter of between about 3.0 cm and about 4.0 cm when in the expanded configuration. The expandable portion of the tissue retractor can have a length of between about 2.0 cm and about 10.0 cm when in the expanded configuration. The expandable portion of the tissue retractor can have a length of between about 2.5 cm and about 5.0 cm when in the expanded configuration. The opening can have a maximum diameter of between about 1.0 cm and about 2.0 cm when in the expandable portion is in the expanded configuration.

In another aspect, this document provides a tissue retractor configured for deployment from a tubular component during a minimally invasive surgical procedure. The tissue retractor comprises, or consists essentially of, a mesh material configured to form at least part of an expandable portion of the tissue retractor, wherein the expandable portion is in a compressed configuration when located within the tubular component, wherein the expandable portion is in an expanded configuration when advanced out of the tubular component, wherein the expanded configuration is capable of retracting tissue at a surgical site during the minimally invasive surgical procedure, wherein the expandable portion defines an opening at a distal end region of the expandable portion, wherein an endoscopic tool is capable of advancing through the expanded region of the expandable portion when the expandable portion is in the expanded configuration and through the opening into contact with tissue at the surgical site. A distal end region of the expandable portion can comprise a structure configured to maintain a desired configuration of the expandable portion. The desired configuration can be a bulb shape. The mesh material can be a two ply mesh material.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a partial cross-sectional view of a portion of an exemplary expandable tissue retractor extending from an exemplary endoscope system.

FIG. 2 is a cross-sectional view of a portion of the tissue retractor of FIG. 1 taken along line A-A in FIG. 1.

FIG. 3 is a cross-sectional view of a portion of the tissue retractor of FIG. 1 taken along line B-B in FIG. 1.

FIG. 4 is a partial cross-sectional view of a portion of an exemplary expandable tissue retractor extending from an exemplary endoscope system.

FIG. 5 is a photograph of three bulb structures.

FIG. 6 is a photograph of an exemplary cage assembly.

FIG. 7 is a model of an inflatable bulb-cap device in an inflated configuration.

FIG. 8 is a model of an inflatable bulb-cap device in a deflated configuration.

FIG. 9 is a front view of the model of an inflatable bulb-cap device of FIG. 7.

FIG. 10 is a photograph of a large glass-made flexible endoscope bulb-shaped cap.

FIG. 11 is a side view of a portion of an expandable tissue retractor in accordance with some embodiments.

FIG. 12 is an end view the expandable tissue retractor shown in FIG. 11.

FIG. 13 is a side view of a portion of an expandable tissue retractor in accordance with some embodiments.

FIG. 14 is a side view of a portion of an expandable tissue retractor in accordance with some embodiments.

FIG. 15 is an expanded view of a portion of the expandable tissue retractor shown in FIG. 14.

FIG. 16 is an end view the expandable tissue retractor shown in FIG. 14.

### DETAILED DESCRIPTION

This document provides methods and materials for retracting tissue during a minimally invasive endoscopic surgical procedure. For example, this document provides methods and materials for using an expandable space-occupying tissue retractor during an endoscopic surgical procedure such as a laparoscopic cholecystectomy procedure, robotic procedure, or a NOTES procedure.

As described herein, a tissue retractor can be configured to be deployed from within a tube (e.g., an outer over-tube) of an endoscope system. Prior to being deployed, a tissue retractor provided herein can be located within the lumen of a tube (e.g., an outer over-tube) in a compressed configuration. Once the distal tip of an endoscope or endoscope component (e.g., an outer over-tube) is in position within a mammal (e.g., a human), the tissue refractor can be advanced in a forward direction. Advancing the tissue retractor in a forward direction can allow an expandable portion of the tissue retractor to extend beyond the one or more tubes (e.g., beyond an outer over-tube) that maintained the expandable portion of the tissue retractor in a compressed configuration. Advancing the tissue retractor in a forward direction also can allow the expandable portion of the tissue retractor to expand to form an expanded configuration. When in the expanded configuration, the tissue retractor can maintain tissue at a surgical site in a desired location or position.

In some cases, a tissue retractor provided herein can include a tubular member that defines a lumen. For example, a tissue retractor provided herein can include a tubular member having an expandable portion at a distal end. In some cases, the expandable portion can be integral with the tubular member or can be removably attached to a distal end of the tubular member. As described herein, the expandable portion of the tissue retractor can be configured to expand to form an expanded configuration, thereby maintaining tissue at the surgical site in a desired location or position. The lumen of a tissue retractor provided herein can be configured such that other surgical tools can be deployed through the lumen to the surgical site. For example, additional endoscope system components such as an endoscope, a surgical tool, a suturing device, a tissue grasping device, and a stapling device can be deployed to the surgical site through the lumen of a tissue retractor provided herein. In some cases, a distal end region of an expandable portion of a tissue retractor provided herein can be configured to define an opening. Such an opening can be designed to allow for the use of surgical tools at the surgical site.

In some cases, 20 or more (e.g., 25, 50, 75, 85, 90, 95, or more) percent of the expandable portion of a tissue retractor provided herein can be transparent. Such a transparent expandable portion of a tissue retractor can allow a user to visualize the tissue being retracted and/or provide a user with a better view of the overall surgical site. In some cases, the entire expandable portion of a tissue retractor provided herein can be constructed of transparent material.

Any type of tissue can be retracted or positioned using a tissue refractor provided herein. For example, visceral, solid organ, vascular, mesenteric, omental, and adipose tissues can be retracted or positioned using a tissue retractor provided herein.

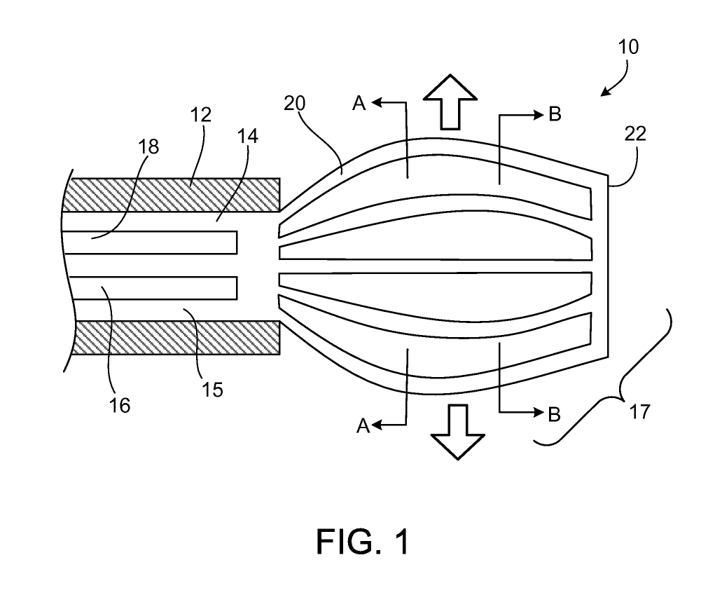

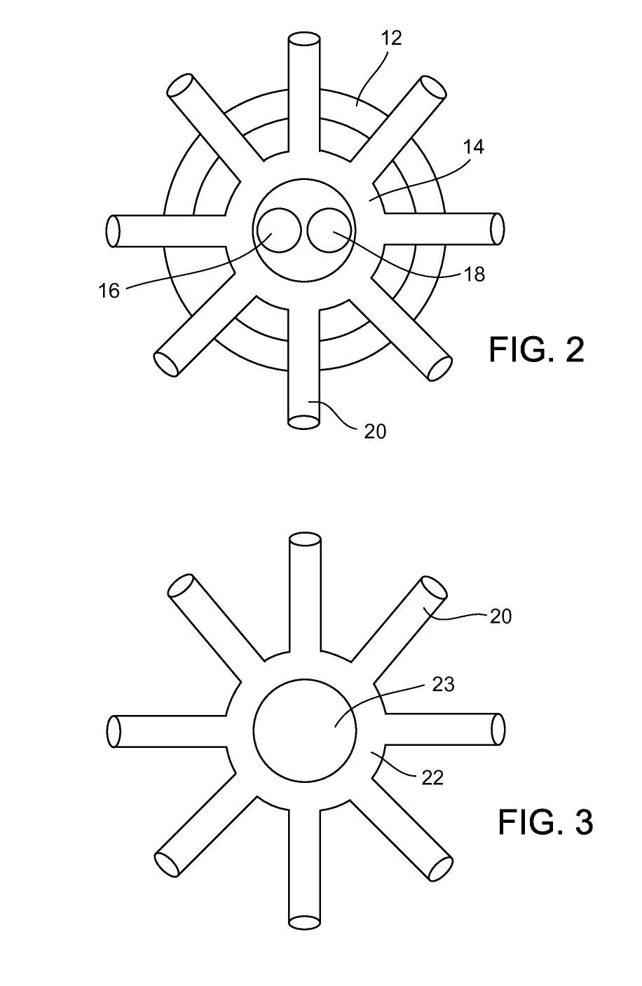

With reference to FIGS. 1, 2, and 3, an endoscopic system 10 can include an outer over-tube 12. Outer over-tube 12 can be configured to have an outer surface that can interface with a patient's internal anatomy and can define an inner lumen. The inner lumen can provide a passageway for other components of an endoscopic system. Endoscopic system 10 can include tissue refractor 14. Tissue retractor 14 can be configured to have a tubular member 15 and an expandable portion 17. In some cases, expandable portion 17 is located at the distal end region of tissue retractor 14. Tissue retractor 14 can be configured to fit within the inner lumen of outer over-tube 12. In some cases, tubular member 15 of tissue retractor 14 can define an inner lumen configured to allow other endoscopic system components such as endoscope 16 and suturing device 18 to be advanced within the inner lumen of tissue retractor 14.

Although only one lumen is depicted in outer over-tube 12 and tubular member 15, it should be understood that multiple lumens can be provided to allow for independent advancement and refraction of other components.

Expandable portion 17 can be configured to include multiple (e.g., two, three, four, five, six, seven, eight, nine, ten, or more) struts 20. Struts 20 can be configured to be expandable such that when expandable portion 17 is no longer retained within outer over-tube 12, they expand radially outward (see, e.g., large arrows of FIG. 1). In some cases, expandable portion 17 can include ring structure 22 attached to one or more of struts 20. Ring structure 22 can define an opening 23 as shown in FIG. 3. Opening 23 can be configured to allow a user to advance surgical tools and other endoscopic system components into contact with a patient's tissue at the surgical site. In some cases, ring structure 22 can be expandable. When in the expanded configuration, struts 20 and ring structure 22 of expandable portion 17 can form a cage-like structure. Examples of materials that can be used to make struts 20 and/or ring structure 22 include, without limitation, linear block copolymers such as polyurethanes, block copolymer of polyethylene terephthalate (PET) and polyethyleneoxide (PEO), thermoplastic polymers (e.g., polynorborene), light activated shape memory polymers metals, polymers, shape memory metals (e.g., nitinol and other shape-memory alloys), and shape memory polymers (e.g., silicone and polytetrafluoroethylene). In some cases, expandable portion 17 of a tissue retractor can be configured to lack ring structure 22.

Struts 20, when deployed outside of the lumen of outer over-tube 12, may not fully expand if they are in an internal body location that constrains their expansion. The force applied by struts 20 on the surrounding tissue may, however, preferably force the tissue outward. Although struts 20 are depicted as expanding generally equally in all directions, it will be understood that they may preferentially expand in only one or more directions. In some cases, although struts 20 are depicted as taking a generally bulb-shape when expanded, it will be understood that they may take a variety of different shapes (e.g., bell-shape, umbrella-shape, round, oval, dome, or wedge (triangular)-shape).

In some cases, expandable portion 17 can expand to form an expanded space region having a maximum diameter of between about 2.5 cm and about 10.0 cm (e.g., 2.5, 3.0, 3.5, 4.0, 4.5, 5.0, 5.5, 6.0, 6.5, 7.0, 7.5, 8.0, 8.5, 9.0, 9.5, or 10.0 cm). In some cases, expandable portion 17 can expand to form an expanded space region having a length between about 2.0 cm and about 10.0 cm (e.g., 2.5, 3.0, 3.5, 4.0, 4.5, 5.0, 5.5, 6.0, 6.5, 7.0, 7.5, 8.0, 8.5, 9.0, 9.5, or 10.0 cm). Such a length can be measured from the point of initial expansion at the proximal end of the expandable portion to the distal most point of the expandable portion that is expanded upon deployment.

The expansion force used to expand expandable portion 17 can be supplied by struts 20, ring structure 22, or a combination thereof. In some cases, struts 20, ring structure 22, or a combination thereof can possess physical characteristics such as elasticity and resiliency that allow expandable portion 17 to be retracted back into the lumen of outer over-tube 12. In some cases, the amount of retraction or advancement can be used to control the size of the expanded configuration of expandable portion 17 during use.

With reference to FIG. 4, expandable portion 17 can be attached to the distal portion of tubular member 15 to form tissue refractor 14. In some cases, tissue retractor 14 can include a layer 24 that is located about struts 20. Layer 24 can be constructed of transparent material (e.g., silicone or a polymer such as polyethylene terephthalate) such that a user can visualize the tissue being retracted and/or can have a better view of the overall surgical site. Layer 24 can have properties of light reflection, properties of light polarization, hydrophilic properties, and/or non-absorptive properties to provide clarity of viewing, atraumatic contact with tissues, and/or frictionless movement over tissues, structures, and organs. In some cases, layer 24 can help maintain retracted tissue at the surgical site in a desired location or position. Layer 24 can extend over, under, or between struts 20. In some cases, layer 24 can extend over some struts, under other struts, and between yet other struts. In some cases, layer 24 can include a coating. Such a coating can be a coating with silica and/or UV inhibitors.

In some cases, an expandable portion can be an expandable synthetic skin. Such a synthetic skin can be made of transparent material (e.g., silicone or a polymer such as polyethylene terephthalate) and expanded using a transparent liquid (e.g., water) to attain a bulb configuration. This skin can have a bulb configuration with a central opening to allow instrumentation, can have a smooth outer surface for easy navigation, can have sufficient rigidity for retracting structures, and/or can fit into a small opening (e.g., a less than 18 mm, less than 17 mm, less than 16 mm, less than 15 mm, less than 14 mm, less than 13 mm, less than 12 mm, less than 11 mm, less than 10 mm, less than 9 mm, less than 8 mm, less than 7 mm, less than 6 mm, or less than 5 mm opening) when deflated.

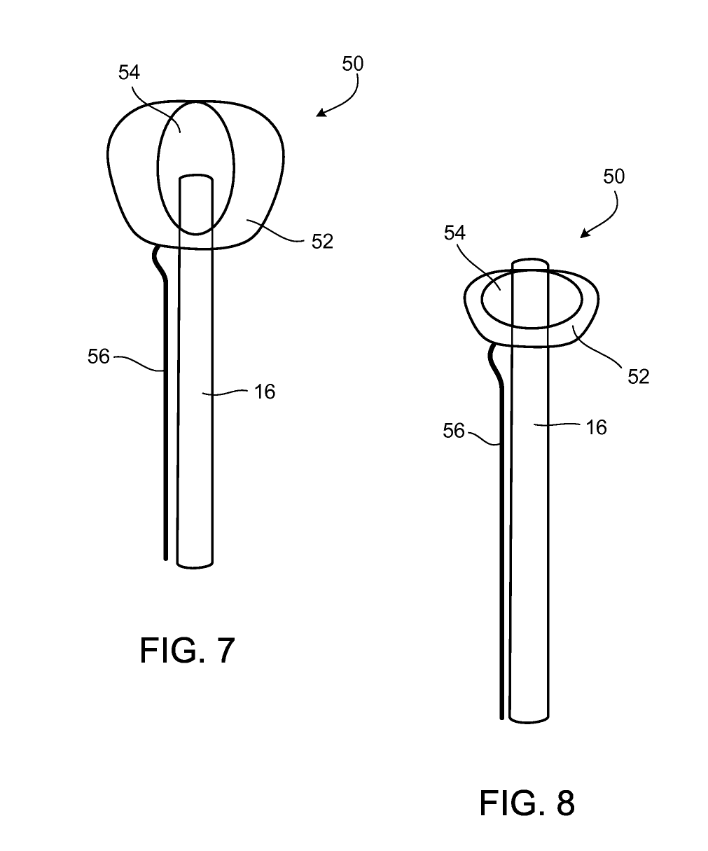

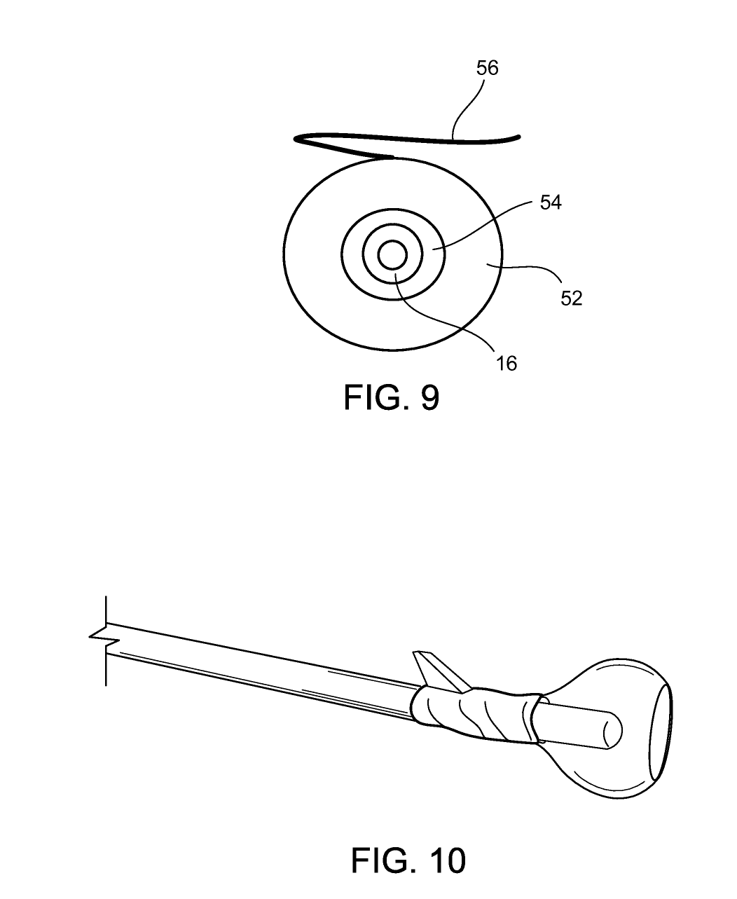

With reference to FIGS. 7-9, endoscopic system 50 includes an expandable portion 52. Expandable portion 52 can be configured to be an expandable synthetic skin that is transparent and that defines a cavity 54. When expanded, expandable portion 52 can have a diameter of between 1.0 cm and 10 cm (e.g., about 2.5, 3.0, 3.5, 4.0, or 4.5 cm) and can have a length between 1.0 cm and 10 cm (e.g., about 2.0, 2.5, 3.0, 3.5, 4.0, or 4.5 cm). Cavity 54 can be configured to allow instruments to be deployed and/or operated at the surgical site. For example, endoscope 16 can be deployed into at least a portion of cavity 54 to provide a surgeon with the ability to view the surgical site. Expandable portion 52 can be configured to expand when inflated with air or a transparent liquid (e.g., water). For example, inflation tube 56 can be configured to inflate expandable portion 52 with air or water. When deflated as shown in FIG. 8, endoscopic system 50 can be withdrawn from a patient.

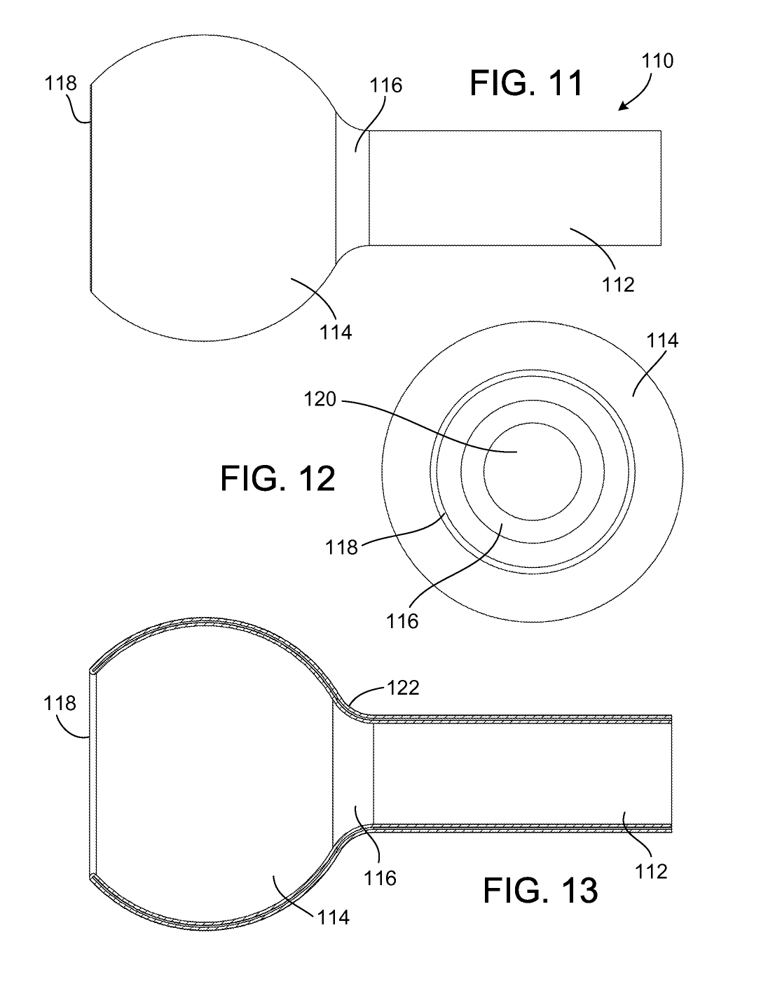

With reference to FIGS. 11, 12, and 13, tissue retractor 110 can be configured to have a tubular member 112 and an expandable portion 114. In some cases, expandable portion 114 can be located at a distal end region of tissue refractor 110. Tissue retractor 110 can be configured to fit within an inner lumen of an outer over-tube that can be used to deliver tissue retractor 110 to a surgical site as described herein. In some cases, tubular member 114 of tissue retractor 110 can define an inner lumen 120 configured to allow other endoscopic system components such as an endoscope and a suturing device to be advanced within inner lumen 120 of tissue retractor 110. In some cases, tissue retractor 110 can include a transition region 116 located between tubular member 112 and expandable portion 114. Transition region 116 can be configured or constructed of a material designed to allow expandable portion 114 to expand while maintaining the tubular configuration of tubular member 112. Tissue retractor 110 can include a distal edge 118 that defines an opening. In some cases, distal edge 118 can be configured to maintain the appropriate configuration of expandable portion 114. In some cases, this opening can provide continuous access from inner lumen 120 of tubular member 112 to a tissue at a surgical site. Any appropriate material can be used to construct the components of tissue retractor 110. For example, a nitinol mesh material can be used to construct all or part of expandable portion 114, transition region 116, and/or tubular member 112. With reference to FIG. 13, expandable portion 114, transition region 116, and tubular member 112 can be constructed of a two-ply nitinol mesh structure 122. In some cases, single ply or two or more ply material (e.g., nitinol material) can be used to construct an expandable portion, a transition region, and/or a tubular member of a tissue retractor. In some cases, such materials can be configured to be transparent. Such a transparent material can allow a user to visualize the tissue being retracted and/or provide a user with a better view of the overall surgical site. In some cases, the entire expandable portion of a tissue retractor provided herein can be constructed of transparent material.

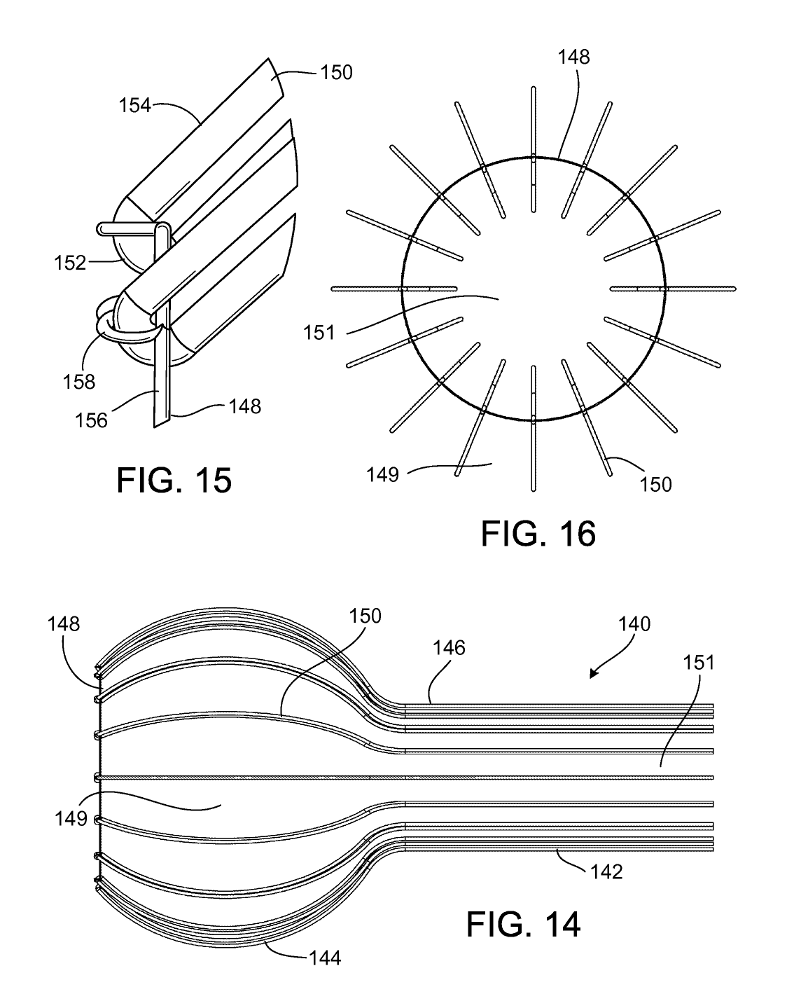

With reference to FIGS. 14, 15, and 16, tissue retractor 140 can be configured to have a tubular member 142 and an expandable portion 144. In some cases, expandable portion 144 can be located at a distal end region of tissue refractor 140. Tissue retractor 140 can be configured to fit within an inner lumen of an outer over-tube that can be used to deliver tissue retractor 140 to a surgical site as described herein. In some cases, tubular member 142 of tissue retractor 140 can define an inner lumen 151 configured to allow other endoscopic system components such as an endoscope and a suturing device to be advanced within inner lumen 151 of tissue retractor 140. In some cases, expandable portion 144 can define an inner open region 149 that is open to inner lumen 151.

In some cases, tissue retractor 140 can include a transition region 146 located between tubular member 142 and expandable portion 144. Transition region 146 can be configured or constructed of a material designed to allow expandable portion 144 to expand while maintaining the tubular configuration of tubular member 142. Tissue retractor 140 can include a positioning structure 148. Positioning structure 148 can define an opening. In some cases, this opening can provide continuous access from inner lumen 151 of tubular member 142 to a tissue at a surgical site.

In some cases, tissue retractor 140 can be constructed of wire structures 150. As shown in FIG. 14, a collection of wire structures 150 can be configured to form expandable portion 144, transition region 146, and/or tubular member 142. In some cases, positioning structure 148 can be configured to position wire structures 150 in a desired position relative to each other. One or more additional positioning structures can be positioned along tissue refractor 140 to help maintain a desired position of wire structures 150. For example, transition region 146 and a proximal end region of tubular member 142 can each include one or more positioning structures designed to allow expandable portion 144 to expand while maintaining the tubular configuration of tubular member 142. Any appropriate structure or material can be used to connect a wire structure (e.g., wire structure 150) to a positioning structure (e.g., positioning structure 148). For example, as shown in FIG. 15, wire structure 150 can include an extending region 154 and a loop region 152. Positioning structure 148 can include an extending region 156 and a ring structure 158. In some cases, extending region 156 can form a particular shape such as large circle as shown in FIG. 16. To connect wire structure 150 to positioning structure 148, wire structure 150 can extend through ring structure 158 and around extending region 156 at or near loop region 152 as shown in FIG. 15. Such a configuration can allow expandable portion 144 to be maintained in a compressed configuration while being advanced within a tubular deployment device and can allow expandable portion 144 to expand to an expanded configuration once the expandable portion 144 is deployed outside a tubular deployment device. In some cases, an expandable portion of a tissue refractor provided herein can be constructed entirely of wire structures and positioning structures (see, e.g., FIGS. 14 and 16). Any appropriate material can be used to construct the components of tissue retractor 140. For example, a nitinol wire material can be used to construct all or part of expandable portion 144, transition region 146, tubular member 142, and/or positioning structure 148. Such an open configuration as shown in FIGS. 14 and 16 can allow a user to visualize the tissue being retracted and/or provide a user with a better view of the overall surgical site. In some cases, the materials used to construct wire structures 150 and positioning structure 148 can be transparent.

The invention will be further described in the following examples, which do not limit the scope of the invention described in the claims.

### EXAMPLES

### Example 1

### Tissue Retractor Sizes and Cage Construction

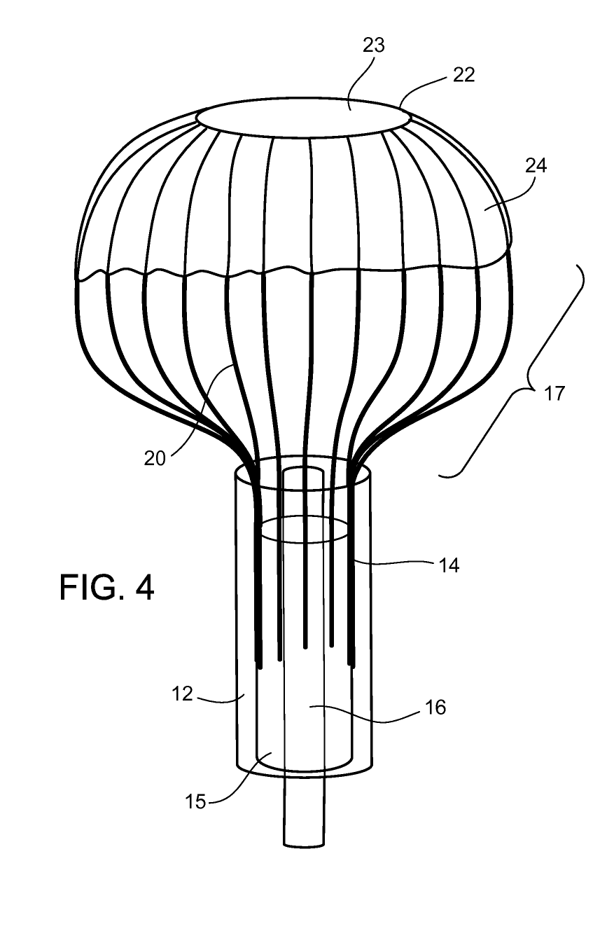

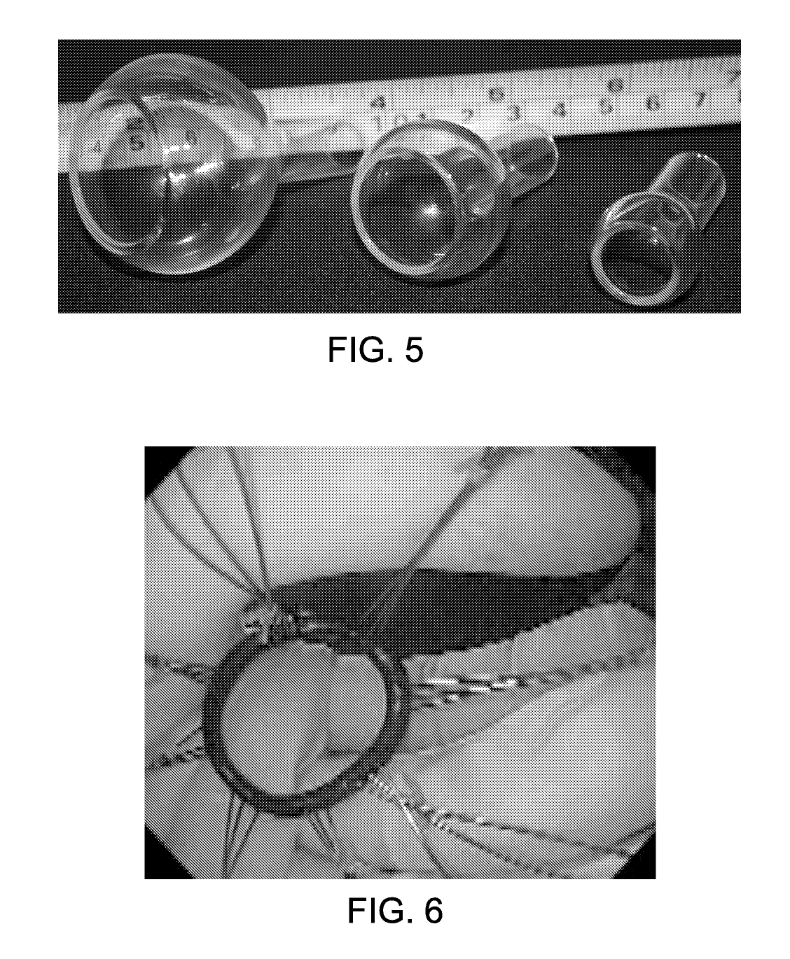

Three different sized bulb-shaped structures were made from glass bulbs (FIG. 5). The three different sized bulb-shaped structures were used to make three different glass-made flexible endoscope bulb-shape caps measuring 4.0 cm diameter×3.0 cm bulb length×3.0 cm outer rim (larger cap); 2.5 cm diameter×3.0 cm bulb length×2.5 cm outer rim (intermediate cap); and 1.8 cm diameter×1.8 cm bulb length×1.6 cm outer rim (smaller cap) by attaching each one at a time to the tip of a flexible endoscope for peritoneal visualization (FIG. 10). Each bulb-shaped structure was tested in human cadavers and pigs. The larger bulb-shaped structure having a 3.5 cm diameter and a 3.0 cm length provided adequate surgical site exposure and allowed for adequate navigation. The smaller sized bulb-shaped structures provided less surgical site exposure and less navigation.

A wire-like cage structure was constructed using nitinol wires and a latex rubber ring (FIG. 6). The wire-like cage structure was tested in live animal abdominal cavities (porcine) and human cadaver abdominal cavities. The wire-like cage structure was capable of atraumatic displacement of solid organs, mesentery, loops of intestine, omentum, and blood vessels.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A tissue retractor configured for deployment from a tubular component during a minimally invasive surgical procedure, wherein said tissue retractor comprises an expandable portion, wherein said expandable portion is in a compressed configuration when located within said tubular component, wherein said expandable portion is in an expanded configuration when advanced out of said tubular component, wherein said expanded configuration is capable of retracting tissue at a surgical site during said minimally invasive surgical procedure, wherein said expandable portion defines an opening at a distal end region of said expandable portion, wherein at least a portion of said expandable portion comprises transparent material configured to allow visualization of at least a portion of the tissue being retracted by said expandable portion, wherein an endoscopic tool is capable of advancing through the expanded region of said expandable portion when said expandable portion is in said expanded configuration and through said opening into contact with tissue at said surgical site.

2. The tissue refractor of claim 1, wherein said tissue retractor comprises struts.

3. The tissue retractor of claim 1, wherein said tissue retractor comprises a ring structure.

4. The tissue refractor of claim 1, wherein said expandable portion of said tissue retractor has a maximum diameter of between about 2.5 cm and about 5.0 cm when in said expanded configuration.

5. The tissue refractor of claim 1, wherein said expandable portion of said tissue retractor has a maximum diameter of between about 3.0 cm and about 4.0 cm when in said expanded configuration.

6. The tissue refractor of claim 1, wherein said expandable portion of said tissue retractor has a length of between about 2.0 cm and about 10.0 cm when in said expanded configuration.

7. The tissue refractor of claim 1, wherein said expandable portion of said tissue retractor has a length of between about 2.5 cm and about 5.0 cm when in said expanded configuration.

8. The tissue refractor of claim 1, wherein said opening has a maximum diameter of between about 1.0 cm and about 2.0 cm when in said expandable portion is in said expanded configuration.

9. A tissue retractor configured for deployment from a tubular component during a minimally invasive surgical procedure, wherein said tissue retractor comprises a plurality of wire structures configured to form at least part of an expandable portion and at least part of a tubular portion, wherein said expandable portion is in a compressed configuration when located within said tubular component, wherein said expandable portion is in an expanded configuration when advanced out of said tubular component, wherein said expanded configuration is capable of retracting tissue at a surgical site during said minimally invasive surgical procedure, wherein said expandable portion defines an opening at a distal end region of said expandable portion, wherein an endoscopic tool is capable of advancing through the expanded region of said expandable portion when said expandable portion is in said expanded configuration and through said opening into contact with tissue at said surgical site.

10. The tissue retractor of claim 9, wherein said tissue retractor comprises a positioning structure configured to maintain the positioning of said plurality of wire structures.

11. The tissue retractor of claim 10, wherein said positioning structure and at least a portion of said plurality of wire structures form the entirety of said expandable portion.

12. The tissue retractor of claim 9, wherein said expandable portion of said tissue retractor has a maximum diameter of between about 2.5 cm and about 5.0 cm when in said expanded configuration.

13. The tissue retractor of claim 9, wherein said expandable portion of said tissue retractor has a maximum diameter of between about 3.0 cm and about 4.0 cm when in said expanded configuration.

14. The tissue retractor of claim 9, wherein said expandable portion of said tissue retractor has a length of between about 2.0 cm and about 10.0 cm when in said expanded configuration.

15. The tissue retractor of claim 9, wherein said expandable portion of said tissue retractor has a length of between about 2.5 cm and about 5.0 cm when in said expanded configuration.

16. The tissue retractor of claim 9, wherein said opening has a maximum diameter of between about 1.0 cm and about 2.0 cm when in said expandable portion is in said expanded configuration.

17. A tissue retractor configured for deployment from a tubular component during a minimally invasive surgical procedure, wherein said tissue retractor comprises a mesh material configured to form at least part of an expandable portion of said tissue retractor, wherein said expandable portion is in a compressed configuration when located within said tubular component, wherein said expandable portion is in an expanded configuration when advanced out of said tubular component, wherein said expanded configuration is capable of retracting tissue at a surgical site during said minimally invasive surgical procedure, wherein said expandable portion defines an opening at a distal end region of said expandable portion, wherein an endoscopic tool is capable of advancing through the expanded region of said expandable portion when said expandable portion is in said expanded configuration and through said opening into contact with tissue at said surgical site.

18. The tissue retractor of claim 17, wherein a distal end region of said expandable portion comprises a structure configured to maintain a desired configuration of said expandable portion.

19. The tissue retractor of claim 18, wherein said desired configuration is a bulb shape.

20. The tissue retractor of claim 17, wherein said mesh material is a two ply mesh material.
